(19) United States
(12) Patent Application Publication
(10) Pub. No.: US 2024/0157091 A1
(43) Pub. Date: May 16, 2024

(54) Navigation Guide for Catheter Tips

(71) Applicant: Toro Neurovascular, Inc., Irvine, CA (US)

(72) Inventors: Hyung Posalit, Irvine, CA (US); Ryan Walker, Costa Mesa, CA (US)

(73) Assignee: Toro Neurovascular, Inc., Irvine, CA (US)

(21) Appl. No.: 18/388,468

(22) Filed: Nov. 9, 2023

Related U.S. Application Data
(60) Provisional application No. 63/424,929, filed on Nov. 13, 2022.

(51) Int. Cl. A61M 25/06; A61M 25/01

(52) U.S. Cl. CPC A61M 25/0662; A61M 25/0108; A61M 25/0138; A61M 2025/0681

(57) ABSTRACT

A navigation tool assembly has a delivery wire having a distal end and a proximal end, a grip connected to the proximal end of the delivery wire, and a guide sleeve having a tapered distal end and a proximal end that is attached to the distal end of the delivery wire, the guide sleeve having a slit that extends from the proximal end of the guide sleeve to the distal end of the guide sleeve. The guide sleeve is used with an outer catheter having a shaft with a lumen extending therethrough, and either a guidewire or a microcatheter extending through the lumen. The guide sleeve is wrapped around the guidewire or the microcatheter and is retained inside the lumen at the distal end of the shaft of the outer catheter.

### BACKGROUND OF THE INVENTION

### 1. Field of the Invention

The present invention is directed to a navigation tool assembly for use in guiding the delivery of a catheter through human vasculature.

### 2. Description of the Prior Art

The ledge effect is often encountered at the origin of the ophthalmic artery when navigating a larger catheter through the carotid siphon. FIGS. 1A-1B illustrate a catheter C that is tracking over a guidewire/microcatheter G and is not able to navigate past a ledge L because the distal end of the catheter C is caught at the ledge L. Currently, reduction of the gap between the larger catheter and the inner catheter or guidewire is considered the first-line approach to mitigating the ledge effect. This can be seen in FIG. 1B as the reduced gap would prevent the distal end of the larger catheter from getting “caught” at the ledge L. This ledge reduction is typically achieved by inserting a medium-sized catheter in between the guidewire/microcatheter G and the catheter C that is caught on the ledge. This reduces the ledge distance to be between the catheter C and the newly-inserted medium-sized catheter. This is burdensome because it typically requires an exchange procedure, which involves removal of guidewire/microcatheter G from within the catheter C in order to insert the medium-sized catheter. When the guidewire/microcatheter G is removed, access to the target lesion is lost. Additionally, once the medium-sized catheter is inserted within catheter C, the guidewire/microcatheter G must be re-inserted into the medium-sized catheter, and must be re-navigated to the lesion site to regain access. These steps add extra procedure time, cost, and pose additional risks to the patient.

### SUMMARY OF THE DISCLOSURE

One of the objects of the present invention is to provide a tracking or navigation tool that enables the catheter to successfully navigate past any ledges that may be encountered and reach the desired location.

To accomplish the objectives set forth above, the present invention provides a navigation tool assembly having a delivery wire having a distal end and a proximal end, a grip connected to the proximal end of the delivery wire, and a guide sleeve having a tapered distal end and a proximal end that is attached to the distal end of the delivery wire, the guide sleeve having a slit that extends from the proximal end of the guide sleeve to the distal end of the guide sleeve. The guide sleeve is used with an outer catheter having a shaft with a lumen extending therethrough, and either a guidewire or a microcatheter extending through the lumen. The guide sleeve is wrapped around the guidewire or the microcatheter and is retained inside the lumen at the distal end of the shaft of the outer catheter.

### BRIEF DESCRIPTION OF THE DRAWINGS

FIGS. 1A and 1B illustrate the ledge effect for a catheter C that is tracking over a guidewire/microcatheter through the carotid siphon.

FIG. 2 is an exploded perspective view of a navigation tool assembly according to one embodiment of the present invention.

FIG. 3 is a schematic view of a conventional catheter system that can be used with the navigation tool assembly of FIG. 2 using a guidewire.

FIGS. 4A-4F illustrate how the tapered distal guide sleeve of the navigation tool assembly of FIG. 2 is inserted through the catheter of FIG. 3.

FIG. 5 illustrates the removal of the guide sleeve from the catheter.

FIG. 6A is a schematic view of a delivery wire surrounded by a coil and a jacket.

FIG. 6B is an enlarged schematic view of the area labeled B in FIG. 6A.

FIGS. 7-10B illustrate different embodiments for wrapping the guide sleeve to create different slits or different degrees of overlaps.

FIG. 11 is a side schematic view of the profile of the guide sleeve of FIG. 2.

FIGS. 12A-12D are schematic side views of different profiles that can be used for the guide sleeve of FIG. 2.

FIG. 13A is a side schematic view of the guide sleeve of FIG. 2.

FIGS. 13B and 13C are schematic views of modifications that can be made to the guide sleeve shown in FIG. 13A.

FIG. 14A is a side schematic view of a guide sleeve according to another embodiment.

FIG. 14B is a top view of the guide sleeve of FIG. 14A.

FIG. 14C is an isometric view of the guide sleeve of FIG. 14A.

FIG. 15 is an isometric view of the guide sleeve of FIG. 2 with the addition of an axial fiber.

FIG. 16 is an enlarged perspective view of the distal end of the guide sleeve of FIG. 2 with the addition of an inner liner.

FIG. 17 is a schematic view of a conventional catheter system of FIG. 3 that can be used with the navigation tool assembly of FIG. 2 using a microcatheter.

FIGS. 18A-18C illustrate how the tapered distal guide sleeve of the navigation tool assembly of FIG. 17 is used with the catheter system and the microcatheter.

FIGS. 19A-19C show one way for the navigation tool assembly of FIGS. 2 to navigate the ledge effect.

FIGS. 20A-20C show another way for the navigation tool assembly of FIG. 2 to navigate the ledge effect.

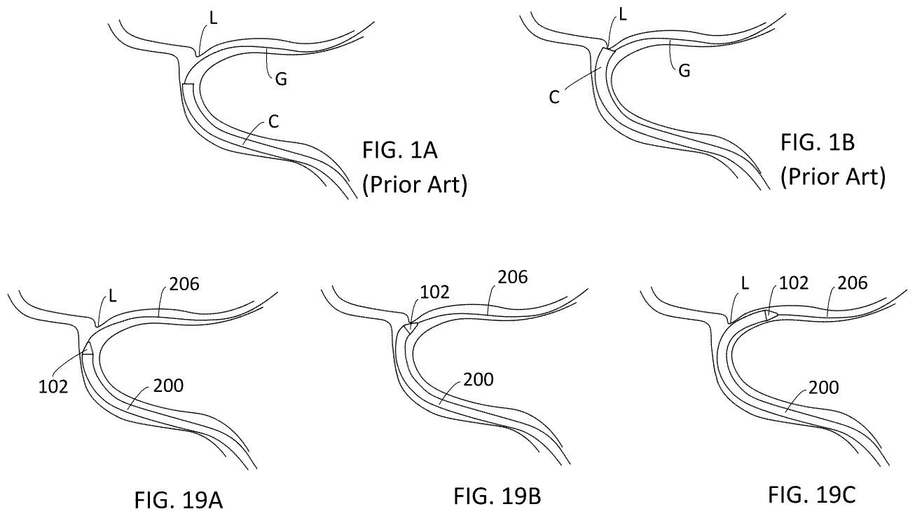

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

The navigation tool assembly of the present invention is a rapid-exchange device that is used to eliminate the ledge effect experienced by catheters, and to enable increased navigability, faster catheter tracking, and improved safety.

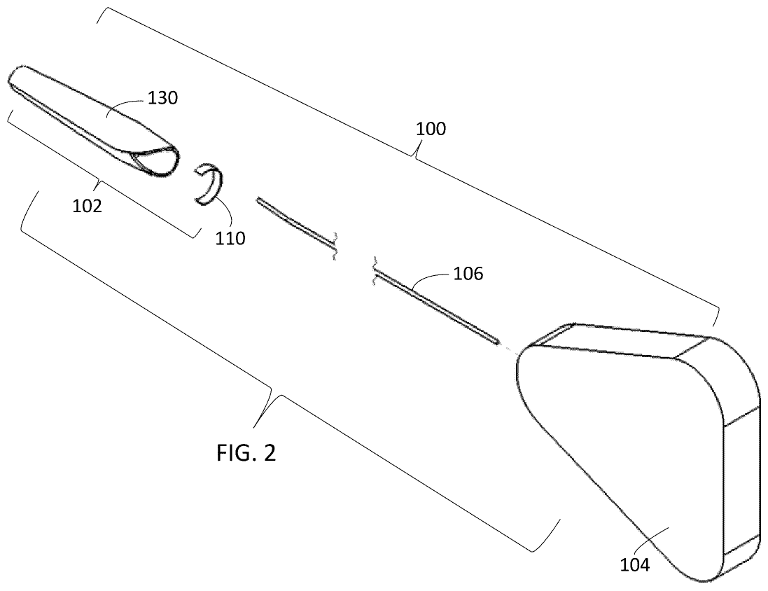

Referring to FIG. 2, the navigation tool assembly 100 has a tapered distal guide sleeve 102, an optional thumb grip 104, and a delivery wire 106 that connects the guide sleeve 102 to the thumb grip 104. The guide sleeve 102 has a sleeve portion 130 and an optional support band 110. The structure of these components, and various embodiments thereof, will be described in greater detail hereinbelow.

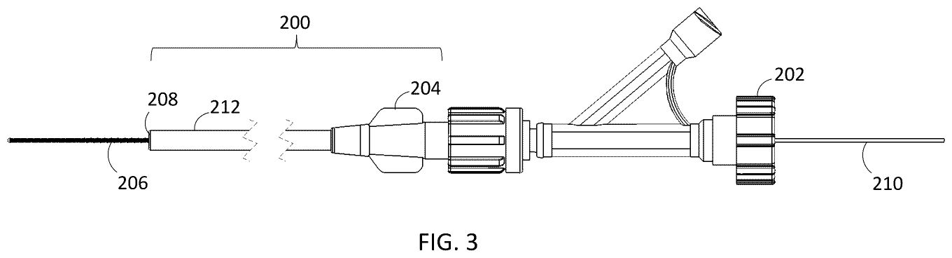

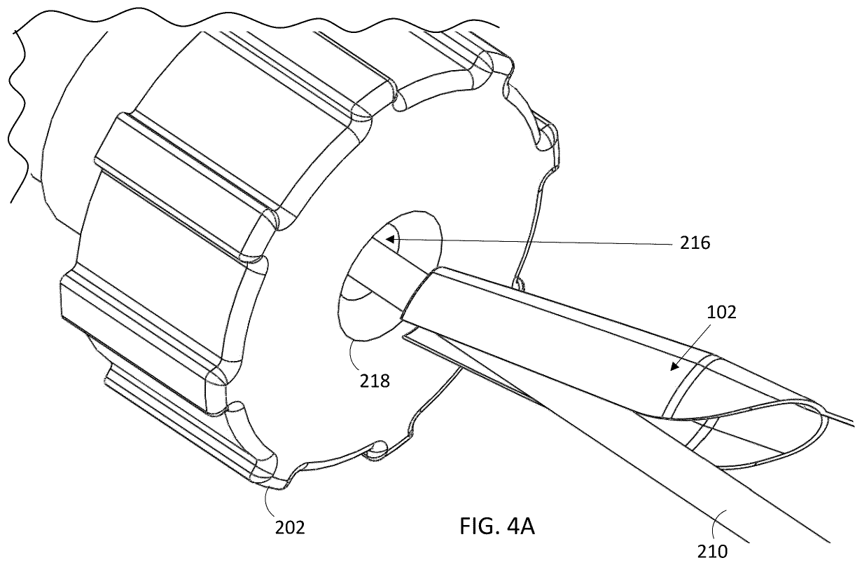

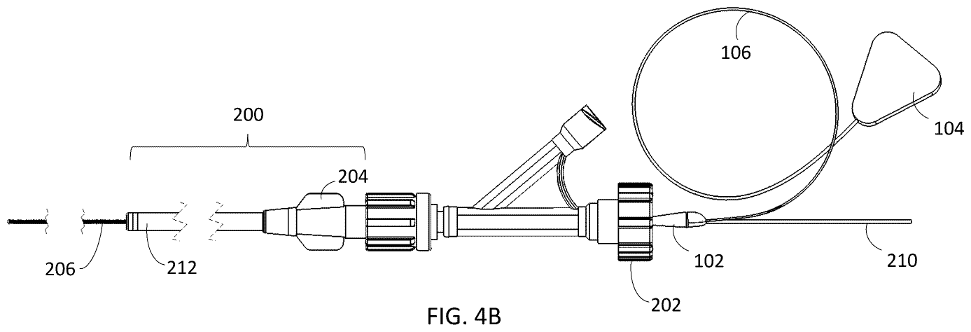

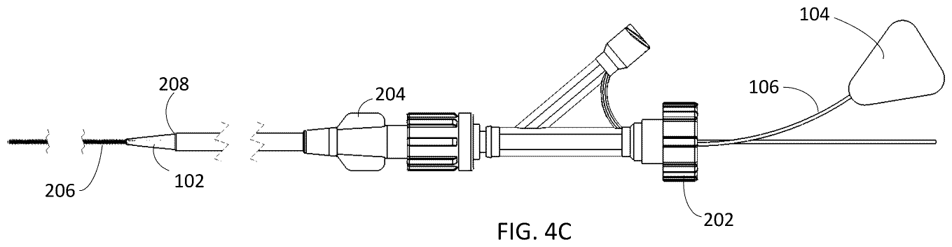

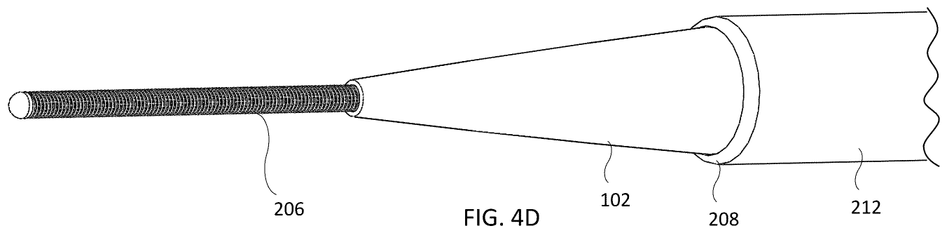

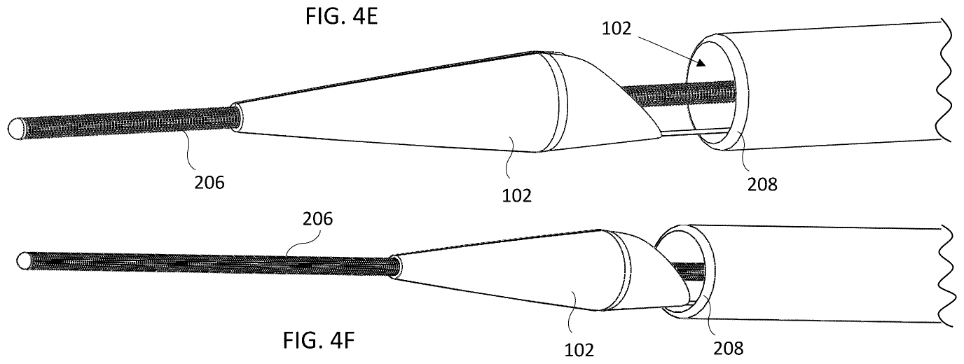

FIG. 3 illustrates an outer catheter 200 that can be used with the navigation tool assembly 100, using a guidewire 206. A standard configuration for the catheter 200 during vascular access includes using a separate rotating hemostasis valve (RHV) 202 attached to the proximal hub 204. A guidewire 206 is loaded inside of the catheter 200, with its distal tip extending past the distal end 208 of the shaft 212 of the catheter 200 and its proximal wire section 210 protruding proximally past the RHV 202. The catheter shaft 212 has at least one lumen 220 (see FIG. 4E) which extends from the distal end 208 to the proximal hub 204, as is typical with conventional catheter shafts.

FIGS. 4A-4E illustrate how the tapered distal guide sleeve 102 of the navigation tool assembly 100 is inserted through the catheter 200 of FIG. 3. The RHV 202 has a bore 216 which communicates with the lumen 220 of the catheter shaft 212 via another bore (not shown) in the proximal hub 204. The guide sleeve 102 is inserted through the proximal opening 218 of the bore 216 (see FIGS. 4A and 4B) by slipping or wrapping it directly over the guidewire 206 (in a coaxial configuration) at the proximal wire section 210, without needing to remove the guidewire 206 from the catheter 200, so that the navigation tool assembly 100 has a rapid exchange loading mechanism. In this regard, the guide sleeve 102 is flexible, allowing it to expand when it is being attached and then contract back down to make a tight fit.

Referring now to FIG. 4C, after attachment, the guide sleeve 102 can be introduced into the RHV 202 and pushed up through the catheter lumen using the delivery wire 106 until the guide sleeve 102 reaches and exits the distal end 208 of the catheter shaft 212. FIG. 4D is an enlarged view of the distal end 208 of FIG. 4C.

As best shown in FIG. 4D, the tapered configuration of the guide sleeve 102 provides a smooth transition between the distal end 208 of the catheter shaft 212 and the guidewire 206 so that the ledge effect is eliminated or significantly reduced. In addition, as shown in FIGS. 4E and 4F, the guide sleeve 102 is fully retractable and self-centering within the catheter shaft 212, so if it gets pushed entirely past the distal end 208, it can be withdrawn back inside the lumen 220. Finally, as shown in FIG. 5, after usage, the guide sleeve 102 can be removed in reverse order. Specifically, the thumb grip 104 and delivery wire 106 are pulled to bring the guide sleeve 102 back and outside of the RHV 202, and then the guide sleeve 102 can be removed from over the guidewire 206 (or microcatheter if a microcatheter is used a guide mechanism instead of a guidewire). Alternatively, the guidewire 206 and navigation tool assembly 100 may be removed from the catheter 200 as a system, with the guide sleeve 102 still loaded over the distal end of the guidewire 206.

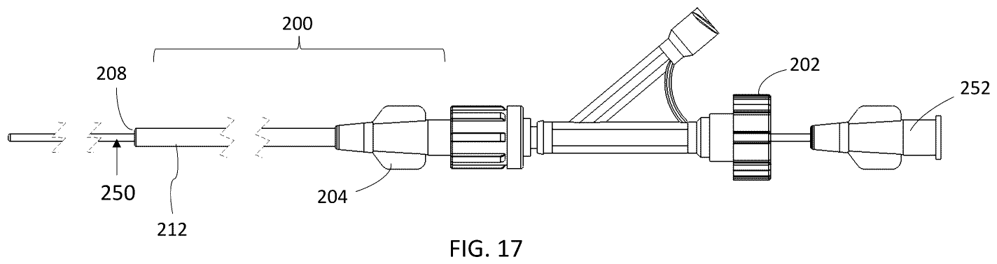

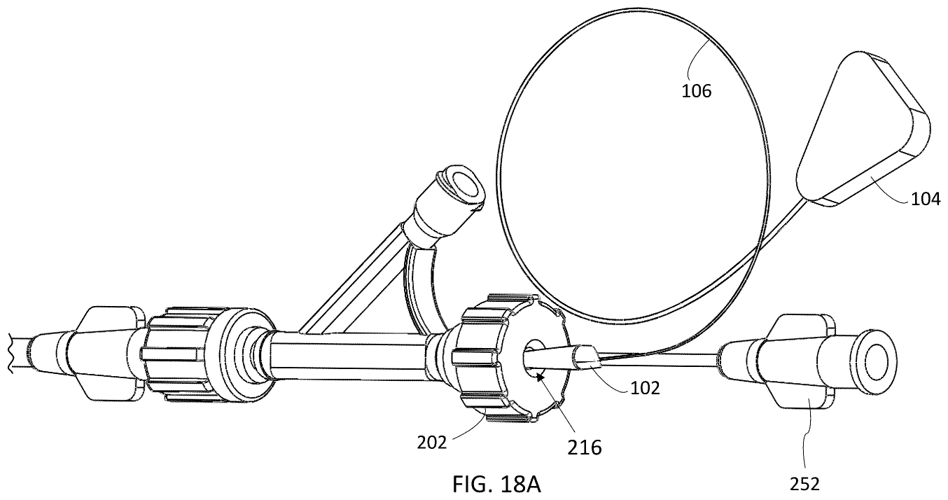

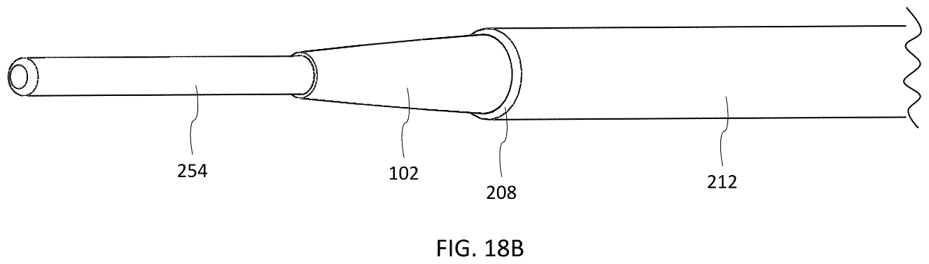

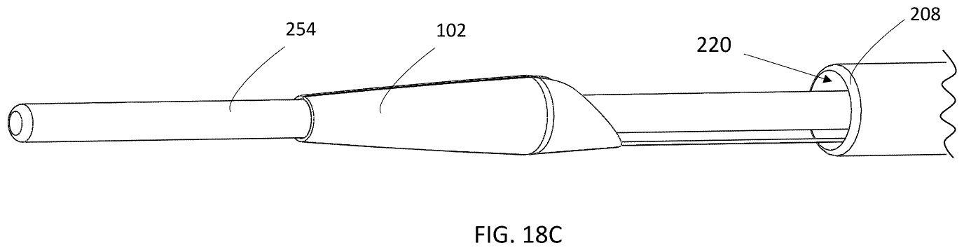

FIG. 17 illustrates the navigation tool assembly 100 in use with the catheter 200, but using a microcatheter 250. FIG. 17 is the same as FIG. 3 except that the guidewire 206 is replaced by a microcatheter 250. The microcatheter has a proximal hub 252 and a catheter shaft 254 (see FIG. 18B). The operation using a microcatheter 250 is the same as with the guidewire 206, so further description will not be provided. FIG. 18A corresponds with FIGS. 4A and 4B to show the guide sleeve 102 inserted through the proximal opening 218 of the bore 216 by slipping or wrapping it directly over the microcatheter 250. FIG. 18B corresponds with FIG. 4D and shows the guide sleeve 102 exiting the distal end 208 of the catheter shaft 212. FIG. 18C corresponds with FIG. 4E to show that the guide sleeve 102 is fully retractable and self-centering within the catheter shaft 212.

Physicians that perform interventional neuroradiology procedures have many device configurations to choose from. Some will utilize the configuration of an outer catheter and a guidewire, and some will use an outer catheter with a microcatheter (with the guidewire usually inside of the microcatheter). Therefore, the navigation tool assembly 100 will either interact with a guidewire or microcatheter in the inner section depending on how the physician has set up the case, and the outer catheter in the outer section.

The structure of the various components of the navigation tool assembly 100 will now be described.

Referring back to FIG. 2, the optional thumb grip 104 attaches (via adhesive, press-fit, or any other commonly used bonding method) to the proximal end of the delivery wire 106 and is used to allow manipulation and handling of the delivery wire 106. The thumb grip 104 may be made of any appropriate polymer or metal and may be designed in any shape or size as appropriate for user-convenience. The size of the thumb grip 104 ranges from approximately 0.5″ in length/width to 2″ in length/width, and 0.05″ to 0.5″ in thickness.

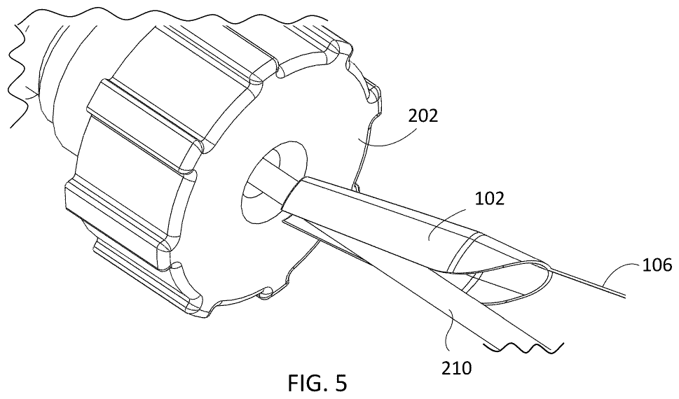

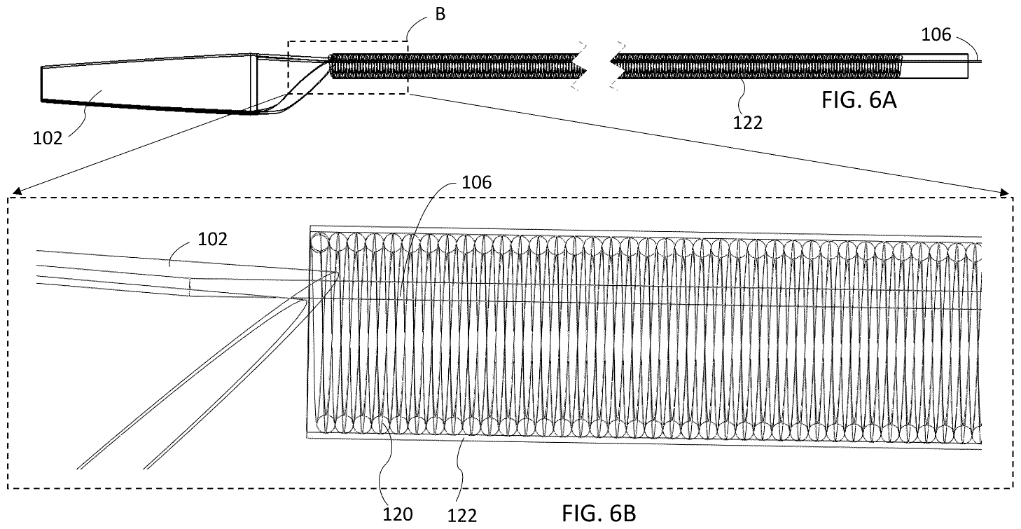

Referring now to FIGS. 6A and 6B, the delivery wire 106 is a tapered wire made of Nitinol, Stainless Steel, or other flexible metal. To enhance pushability, in some embodiments, a partial or full length of the delivery wire 106 may be surrounded by a coil 120 that is made of Stainless Steel, Nitinol, Platinum, or other metallic wire. The purpose of the coil is to create a larger diameter over the delivery wire 106, such that the bendability of the delivery wire 106 inside of the catheter shaft 212 is reduced. A thin polymer jacket 122 can be provided over the coil 120 and/or the delivery wire 106 for reduced friction. For example, the jacket 122 can be made from PTFE, Nylon, HDPE, or any polymer doped with a lubricious additive. In the embodiment shown in FIGS. 6A and 6B, the distal end of the delivery wire 106 is supported by both a coil 120 and a jacket 122.

The delivery wire 106 may taper between diameters of 0.002″ to 0.040″, typically going from a thin distal end to a thicker proximal end (e.g., 0.006″ distal end, tapering to 0.010″ mid-section, then tapering to 0.020″ proximal section).

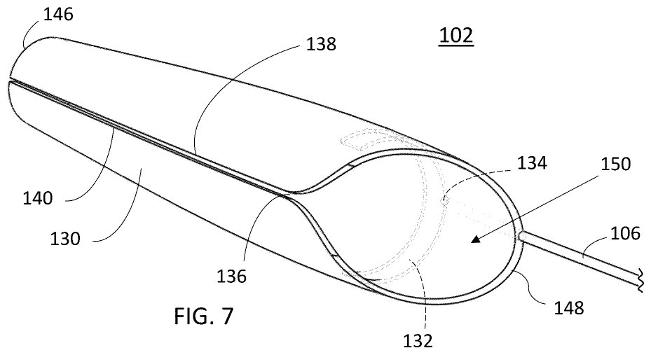

The distal end of the delivery wire 106 is connected to the guide sleeve 102. FIG. 7 illustrates one embodiment of a guide sleeve 102. The guide sleeve 102 has a shaped polymer sleeve portion 130 and a metallic support band 132. The metallic support band 132 is used to attach to the delivery wire 106. If the support band 132 is omitted, the delivery wire 106 would be embedded within the guide sleeve 102. The delivery wire 106 can be embedded in the proximal portion, or may be embedded from end to end within the guide sleeve 102.

In the embodiment of FIG. 7, the support band 132 is embedded inside the sleeve portion 130 and is attached at attachment location 134 to the distal end of the delivery wire 106 via welding (e.g., laser, vibration), adhesive, crimping, or any other typically used mechanism of attachment between metals. The sleeve portion 130 can be made from a soft, elastic polymer such as a polyurethane or PEBA. The polymer may be doped with additives to increase lubricity, add radiopaque visibility, or increase chemical stability. It may contain a PTFE liner on the inner surface and may contain a hydrophilic coating on the outer and/or inner surface, as described below.

The sleeve portion 130 is essentially a flat sheet of material that is shaped to be curled by about 360 degrees. In the embodiment of FIG. 7, a slit 136 runs axially parallel to the longitudinal axis along the length of the sleeve portion 130, and is defined by the opposite edges 138 and 140 of the sheet of material. The slit 136 extends from the distal end 146 of the sleeve portion 130 to the proximal end 148 of the sleeve portion 130. This configuration enables the sleeve portion 130 to wrap around (or clip over) a microcatheter or guidewire before being loaded into the catheter 200 as shown in FIGS. 4A-4F and 18A, and to adjust its internal size or volume as it is slid through the lumen 220 of the catheter 200. The slit 136 is what enables the guide sleeve 102 to be a “rapid exchange system”, as neither the catheter 200 nor the guidewire 206 need to be removed while loading the guide sleeve 102. The embodiments below will illustrate different variations to the slit.

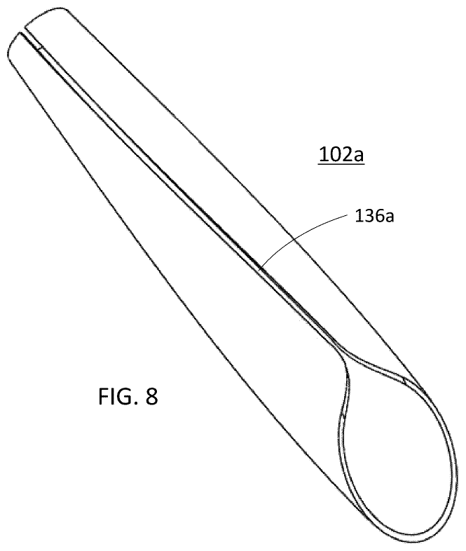

For example, FIG. 8 illustrates a guide sleeve 102a having an axial slit 136a running at an angle that is not parallel to the longitudinal axis. In FIG. 8, the slit 136a can be between 0-60 degrees along the length of the sleeve portion 130a, and in particular, FIG. 8 shows a 5-degree slit. Otherwise, the guide sleeve 102a can be the same as the guide sleeve 102.

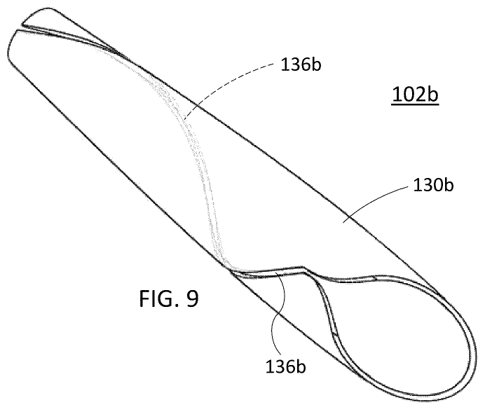

FIG. 9 illustrates a guide sleeve 102b having an axial slit 136b that runs at an angle between 0-60 degrees with respect to the longitudinal axis along the length of the sleeve portion 130b. In particular, FIG. 9 shows a 45-degree slit that essentially winds in a spiral manner from the distal end of the sleeve portion 130b to the proximal end of the sleeve portion 130b.

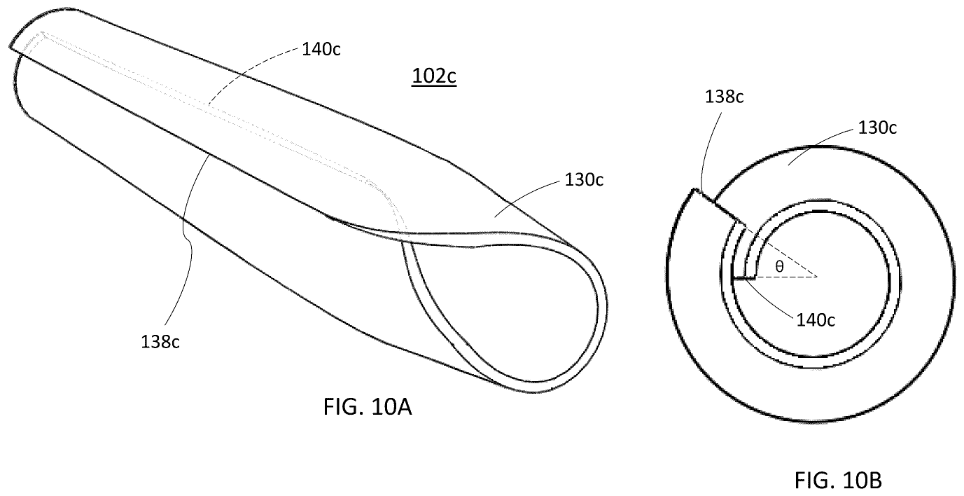

FIGS. 10A and 10B illustrate a guide sleeve 102c that has an overlap. Specifically, the sleeve portion 130c is wound so that one edge 140c overlaps the other edge 138c in a manner such that the overlapped edge 138c is inside the sleeve portion 130c adjacent the overlapping edge 140c. The overlap can be defined by an angle θ as shown in FIG. 10B, and this angle can be between 1 degree to 180 degrees. FIG. 10B shows an overlap of about 30 degrees. This overlap allows the sleeve portion 130c to expand and contract in the radial direction as necessary without losing its grip on the guidewire 206 or microcatheter 250. For overlapping sleeve portions, there is no slit in that there is no space between two edges 138c and 140c in the same plane, but there would be a gap between the layers of the overlapping edges 138c and 140c.

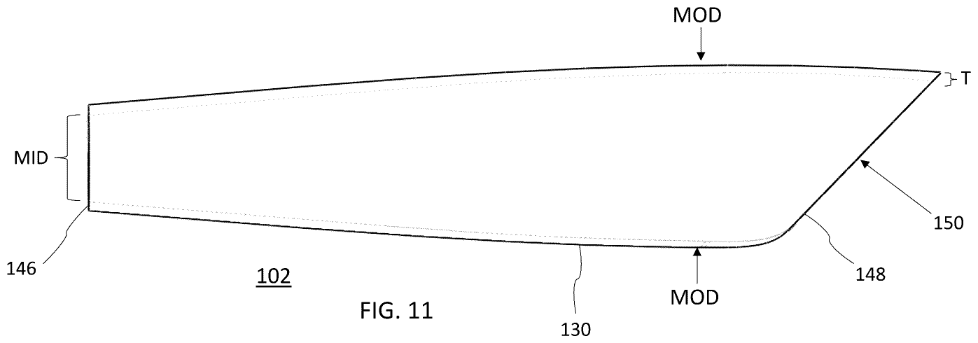

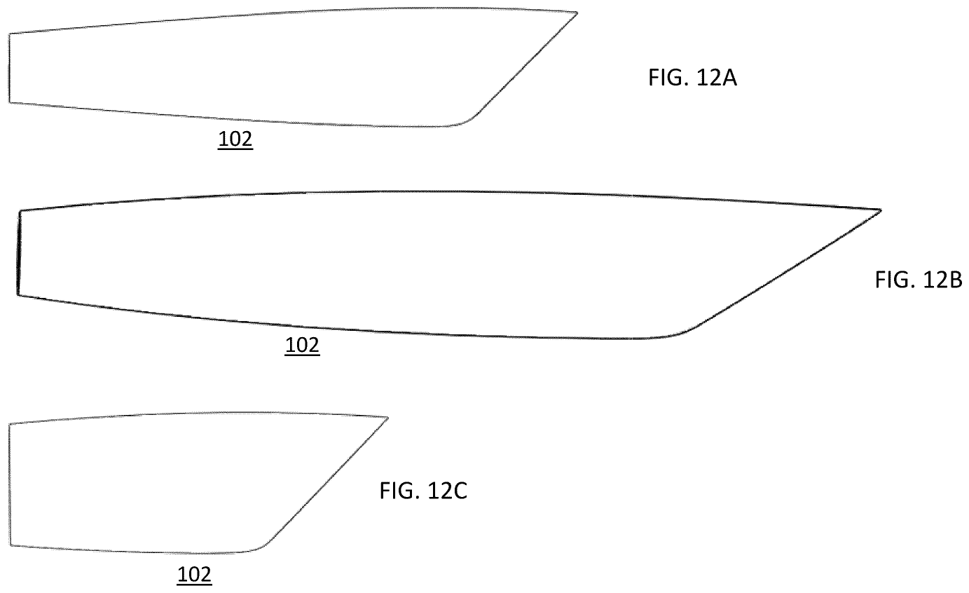

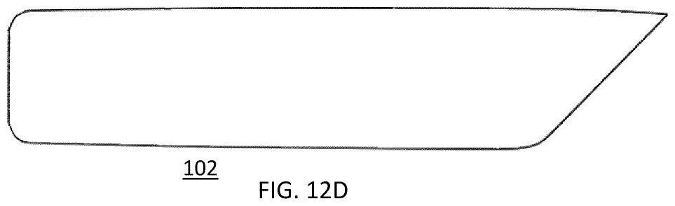

While FIGS. 7-10 show different embodiments relating to the wrapping of the sleeve portion 130 to create different slits or overlaps, FIGS. 11-12D will illustrate the different configurations for the sleeve portions 130.

Starting first with FIG. 11, this FIG. shows the side profile for the guide sleeve 102 in FIG. 2. To better define the various profiles, there are two terms that shall be used. First, a “major OD” (MOD) means the largest outer diameter measurement of the sleeve portion 130. The MOD is usually provided near the center which is intended to fit with minimal clearance (between 0.0001″ to 0.020″) in the lumen 220 of the catheter 200. Second, a “minor inner diameter” is the smallest inner diameter measurement located at the distal end of the sleeve portion 130. The MID and MOD are used to define a taper that is created towards the distal end of the sleeve portion 130.

Referring to FIG. 11, the guide sleeve 102 has two tapers, a first small taper at the proximal end 148 with a smooth transition to the delivery wire 106, which allows for retraction of the guide sleeve 102 back into the lumen 220 if needed, and a second greater taper at the distal end 146 that creates another transition to the delivery wire 106. The MID at the distal end 146 may be flexible and variable, allowing it to conform down to a range of diameters and thus being able to fit tightly over multiple different sizes of guidewires or microcatheters. The proximal end 148 may contain a connector piece (not shown) that can be used as an attachment region between the shaped polymer and the delivery wire 106. The proximal end 148 may have an angled opening 150. Finally, the sleeve portion 130 can have a wall thickness T ranging from 0.001″ to 0.100″.

FIGS. 12A-12C show guide sleeves that have the same general configuration as guide sleeve 102 in FIG. 11, except that they have different lengths. For example, the end-to-end lengths of the guide sleeves 102 can range between 5 mm to 100 mm. FIGS. 12A-12C show both different lengths from the MOD to the distal end 146 and from the MOD to the proximal end 148. Depending on the catheter 200 and the desired anatomy/usage, different lengths may be required to enable navigability through tortuosity, ease-of-use in positioning by the user, or functional effectiveness of the taper and its ability to pull the catheter 200 off the ledge.

FIG. 12D shows a tip which does not have a gradual taper from the MOD to the distal end 146, but instead is mostly a constant diameter from the MOD to the proximal end 148, with a rounding or sharp taper at the distal end 146. This profile is more similar to the profile of the standard medium-sized catheters that are currently used to overcome the ledge effect.

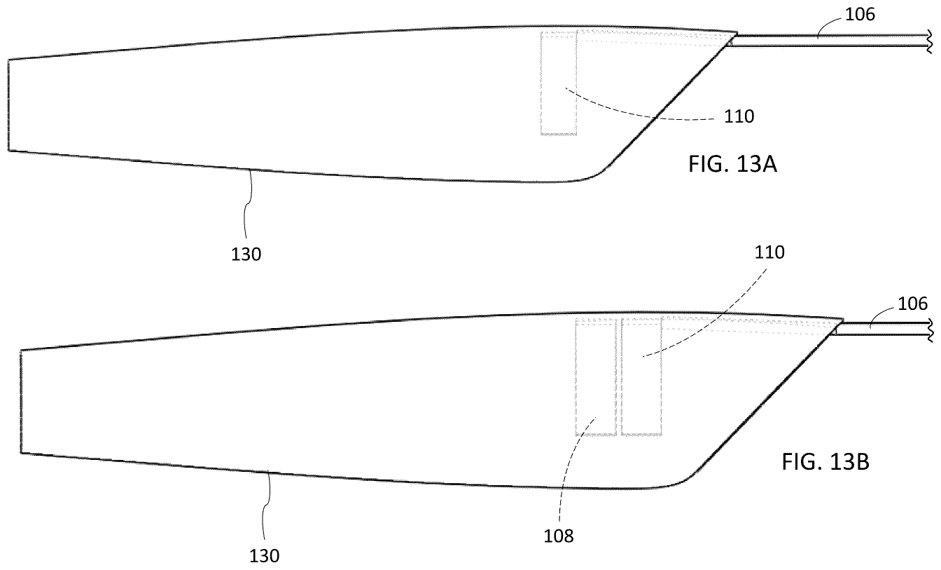

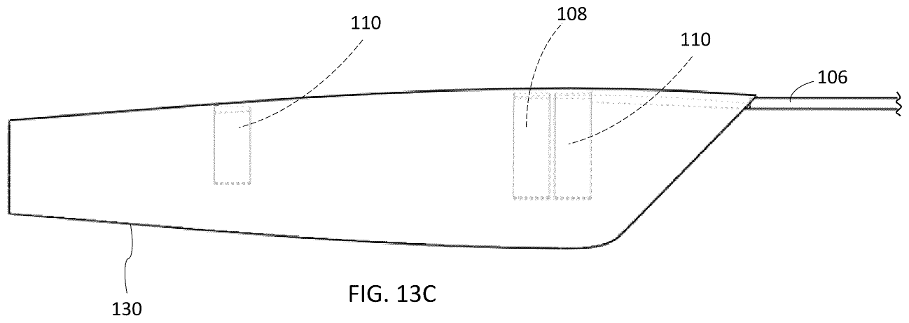

One or more support bands 110 may be embedded within the shaped polymer sleeve portion 130. In one embodiment shown in FIG. 13A (which is the same as that in FIG. 2), one metallic support band 110 is embedded in the sleeve portion 130 adjacent the proximal end 148, and may be made of a radiopaque material such as a Platinum/Iridium alloy, Gold, Tungsten, or similar material, in order to provide visibility under X-Ray. The support band 110 can also be made of a metal with greater mechanical strength such as Stainless Steel or Nitinol, which helps provide structural stability to the sleeve portion 130. In such a case, the sleeve portion 130 may be doped with a radiopaque substance such as Barium Sulfate to provide visibility. The support band 110 is not a full ring; as best shown in FIG. 2, it has a slit to allow the support band 110 and the sleeve portion 130 to snap over a microcatheter or guidewire.

It is also possible to provide a separate marker band 108 in addition to the support band 110. Referring to FIG. 13B, a separate embedded marker band 108 may be added next to the support band 110. The support band 110 is made of Stainless Steel or Nitinol, which helps provide structural stability to the device. The marker band 108 is made of a radiopaque material such as a Platinum/Iridium alloy, Gold, Tungsten, or similar in order to provide visibility under X-Ray. One of the bands 108 or 110 is used to attach the sleeve portion 130 to the delivery wire 106. Neither band 108 and 110 is a full ring and each contains a slit, as described above.

FIG. 13C illustrates another embodiment where a distal support band 110 is provided in addition to the bands 108 and 110 in FIG. 13B. In this embodiment, the additional structure from the two support bands 110 helps to keep the sleeve portion 130 closed to hold the microcatheter or guidewire within the inner diameter.

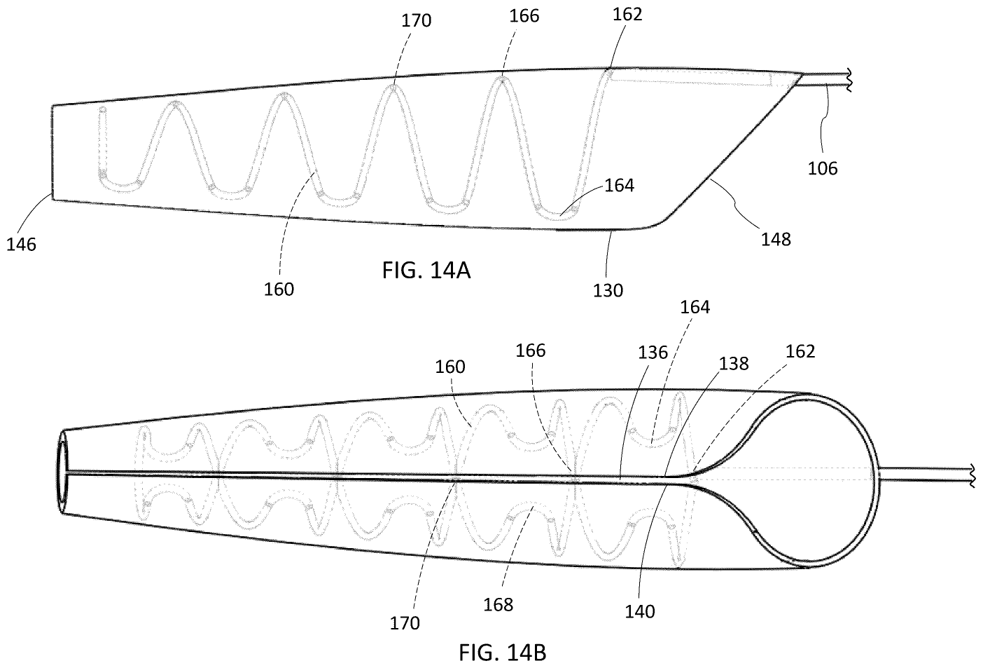

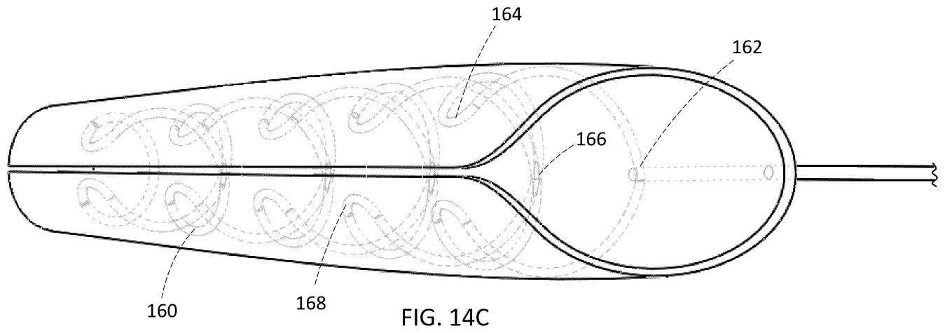

FIGS. 14A-14C are three different views of another embodiment of the guide sleeve 102 where a wire frame 160 can be embedded inside the sleeve portion 130. The wire frame 160 can be provided in lieu of, or in addition to, the support band 110.

The wire frame 160 is created using a metallic wire that is made of a flexible material, such as Nitinol or Stainless Steel, or a combination of other metals, such as “drawn-filled tubing” (where the core of the wire is Nitinol, Gold, Platinum, Tantalum, or Palladium, and the outer wire tubing is Nitinol or other metallic alloy). The wire frame 160 does not cross over the slit 136, thus the slit remains open and unobstructed.

The wire of the wire frame 160 may be wrapped in any configuration along the length of the sleeve portion 130 in order to provide structural support and, if applicable, radiopacity. The wire is fixed in the desired configuration and then heat set to this shape prior to being fused inside the sleeve portion 130, thus providing structural integrity to the sleeve portion 130. This embodiment may also contain marker bands (not shown) similar to the marker band 108, or the sleeve portion 130 may be doped with a radiopaque additive.

The wire for the wire frame 160 can be connected to the delivery wire 106 at the location 162 near the proximal end 148, and then the wire extends in a wavy somewhat sinusoidal manner with peaks and valleys. Specifically, from the location 162, the wire extends to a peak 164 adjacent the edge 138 of the slit 136, and then extends into a valley 166, and then to another peak 168 adjacent the opposite edge 140 of the slit 136, then back to another valley 170, and so on until the wire reaches the distal end 146. The wire may terminate within the sleeve portion 130 near the distal end 146 or, as shown in FIGS. 14A-14C, the wire may return towards the proximal end 148 in the same wavy pattern where the peaks alternate adjacent the edges 138 and 140, and where all the valleys are aligned along the same longitudinal line as the delivery wire 106. In this case, the wire terminates at or near the same initial location 162.

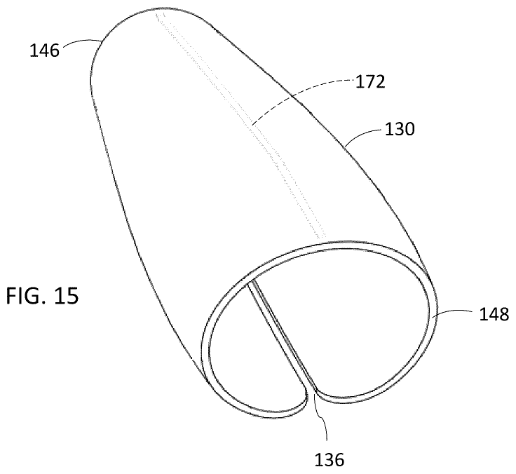

Referring now to FIG. 15, an axial fiber 172 may be embedded inside the sleeve portion 130 to add tensile strength along the length thereof. The axial fiber 172 can extend from the proximal end 148 to the distal end 146 in a straight line. The axial fiber 172 may be made of aramid fiber, carbon fiber, fiberglass, Nylon, rubber, or another similar appropriate material.

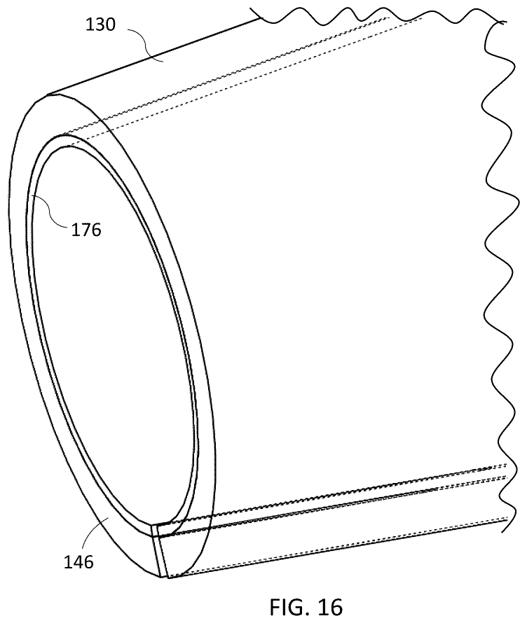

Referring now to FIG. 16, an annular liner 176 may be provided on the inner surface of the sleeve portion 130 to provide lubricity along the inner surface. The liner 176 can be made of PTFE, Nylon, HDPE, or another lubricious polymer. FIG. 16 shows the distal end 146 of the sleeve portion 130, but the liner 176 can extend all the way to the proximal end 148 as well.

In addition, the outer surface, the inner surface, or both, of the sleeve portion 130 may be coated with a hydrophilic coating (not shown) to reduce friction.

Although different embodiments of the slits, support bands 110, marker bands 108, wire frame 160, axial fiber 172 and liner 176, as well as different profiles for the guide sleeve 102, are described hereinabove, it will be appreciated by one skilled in the art that any combination of these components can be used. For example, it is possible to provide a guide sleeve 102 having all of the bands 108, 110, the wire frame 160, the axial fiber 172 and the liner 176 in a selected profile, or in any combination that excludes one or more of these components.

FIGS. 19A-19C illustrate how the navigation tool assembly 100 avoids the ledge effect. In FIG. 19A, the guidewire 206 has traversed the ledge L. In FIG. 19B, further advancement of the catheter 200 will cause the guide sleeve 102 to contact the ledge L. The tapered surface of the guide sleeve 102 provides a smooth transition between the guidewire 206 and the catheter shaft 212, so that the combined guide sleeve 102 and catheter 200 passes the ledge L (see FIG. 19C).

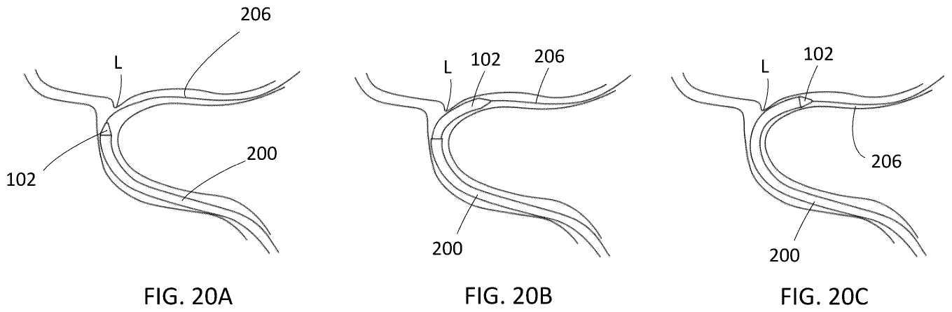

FIGS. 20A-20C illustrate another way of using the navigation tool assembly 100 to avoid the ledge effect. The tapered guide sleeve 102 may be advanced past the ledge L while the guidewire 206 and catheter 200 are held in place. Compare FIGS. 19B and 20B. Once the guide sleeve 102 has reached the target location, the guide sleeve 102 and the guidewire 206 may be held in place while the catheter 200 is advanced over the guide sleeve 102. See FIGS. 20B and 20C.

The structure and configuration of the guide sleeve 102 is critical to the effectiveness of the navigation tool assembly 100 in navigating past a ledge L. The guide sleeve 102 preferably has a tapered configuration where the smallest diameter is at the distal end to provide a smooth transition between the guidewire 206 and the catheter shaft 212. In addition, the slit 136 or the overlap shown in FIG. 10 enables the sleeve portion 130 to wrap around (or clip over) a microcatheter or guidewire before being loaded into the catheter 200 as shown in FIGS. 4A-4F and 18A, and to adjust its internal size or volume as it is slid through the lumen 220 of the catheter 200.

While the description above refers to particular embodiments of the present invention, it will be understood that many modifications may be made without departing from the spirit thereof. The accompanying claims are intended to cover such modifications as would fall within the true scope and spirit of the present invention.

1. An assembly, comprising:
an outer catheter having a shaft with a lumen extending therethrough, and a distal end;
a guidewire or a microcatheter extending through the lumen;
a navigation tool assembly having:
a delivery wire having a distal end and a proximal end;
a guide sleeve having a tapered distal end and a proximal end that is attached to the distal end of the delivery wire, the guide sleeve having a slit that extends from the proximal end of the guide sleeve to the distal end of the guide sleeve; and
wherein the guide sleeve is wrapped around the guidewire or the microcatheter and is retained inside the lumen at the distal end of the shaft of the outer catheter.

2. The assembly of claim 1, wherein a support band is provided in the guide sleeve and connected to the distal end of the delivery wire.

3. The assembly of claim 1, wherein the navigation tool assembly further includes a grip connected to the proximal end of the delivery wire.

4. The assembly of claim 1, wherein a marker band is provided in the guide sleeve.

5. The assembly of claim 1, wherein the tapered distal end has the smallest diameter of any portion of the guide sleeve.

6. The assembly of claim 5, wherein the proximal end of the guide sleeve is tapered.

7. The assembly of claim 1, wherein at least a portion of the delivery wire is surrounded by a coil.

8. The assembly of claim 1, wherein a frame member is provided along the length of the guide sleeve.

9. The assembly of claim 1, wherein the guide sleeve defines a longitudinal axis and the slit is angled with respect to the longitudinal axis.

10. The assembly of claim 1, wherein an axial fiber is provided along a portion of the length of the guide sleeve.

11. The assembly of claim 1, wherein the guide sleeve has an inner surface, and an annular liner is provided on the inner surface of the guide sleeve.

12-21. (canceled)

22. A method of avoiding a ledge effect while delivering an outer catheter through a vasculature, comprising the steps of:
providing an outer catheter having a shaft with a lumen extending therethrough, and a distal end;
extending a guidewire or a microcatheter through the lumen and into a part of the human vasculature adjacent a ledge;
providing a navigation tool assembly having:
a delivery wire having a distal end and a proximal end; and
a guide sleeve having a tapered distal end and a proximal end that is attached to the distal end of the delivery wire;
wrapping the guide sleeve around the guidewire or the microcatheter and retaining the guide sleeve inside the lumen at the distal end of the shaft of the outer catheter;
advancing the outer catheter to a ledge;
advancing the guidewire or microcatheter beyond the ledge; and
using the tapered distal end of the guide sleeve as a transition to guide the outer catheter past the ledge.

23. The method of claim 22, further including providing the guide sleeve with a slit that extends from the proximal end of the guide sleeve to the distal end of the guide sleeve so that the guide sleeve can wrap around the guidewire or the microcatheter.
